(19) United States
(12) Patent Application Publication
(10) Pub. No.: US 2009/0157185 A1
(43) Pub. Date: Jun. 18, 2009

(54) Prosthetic Monolithic Spinal Discs and Method of Customizing and Constructing Discs

(76) Inventor: Chong Chol Kim, Los Angeles, CA (US)

Correspondence Address: SoCAL IP LAW GROUP LLP, 310 N. WESTLAKE BLVD. STE 120, WESTLAKE VILLAGE, CA, 91362, US

(21) Appl. No.: 11/959,437

(22) Filed: Dec. 18, 2007

(51) Int. Cl. A61F 2/44; A61B 17/04

(52) U.S. Cl. 623/17.16; 623/17.11; 623/17.13; 606/74

(57) ABSTRACT

The process uses micro-machining for forming a prosthetic spinal disc in which one of more springs bias adjacent vertebrae apart and absorb shock. Instead of using conventionally wound springs, the entire prosthesis including the springs are formed by micro-machining a block of material. Imaging, load and other patient data provide the parameters for the prosthesis.

### BACKGROUND

1. Technology Field

Prosthetic devices used to replace diseased or damaged spinal discs and, the process for customizing and constructing the discs.

2. General Background and State of the Art

The adult spine has 26 vertebrae (depending how one counts) with fibrocartilage, intervertebral discs between adjacent vertebrae. The vertebrae include seven cervical vertebrae in the neck, twelve thoracic vertebrae below the neck, five lumbar vertebrae for the lower back, one sacrum below the lumbar region and one coccyx, or tailbone. The discs form strong joints, separate, cushion and allow flexure and torsion between the vertebrae.

When functioning properly, the vertebrae and discs allow a person to twist and to bend forward, backward and to the sides. To accomplish this, the discs permit adjacent vertebrae six degrees of motion: vertical (compressing to absorb shock and tension), bending forward and backward, bending to the sides. The discs also permit torsional movement.

### BRIEF DESCRIPTION OF THE DRAWINGS

FIG. 1 is a background drawing showing a side view of the human spine.

FIG. 2 is a background drawing showing a side view of three adjacent vertebrae.

FIG. 3 is a side sectional view of portions of two adjacent vertebrae that shows the intervertebral disc between the vertebrae.

FIG. 4 is a background, perspective drawing showing a representation of the intervertebral disc components.

FIGS. 5 and 6 are side sectional views two adjacent vertebrae with natural discs.

FIG. 5 shows the disc under normal load, and FIG. 6 shows the disc compressed under a heavier load.

FIG. 7 is a perspective view of another prosthetic spinal disc embodiment and is disclosed in applicant's related application Ser. No. 11/027,728.

FIGS. 8, 9, 10 and 11 are perspective views showing one process for making applicants' prosthetic spinal disc.

FIG. 12 is a plan view of a vertebra with two spaced discs.

### DETAILED DESCRIPTIONS OF EXEMPLARY EMBODIMENTS

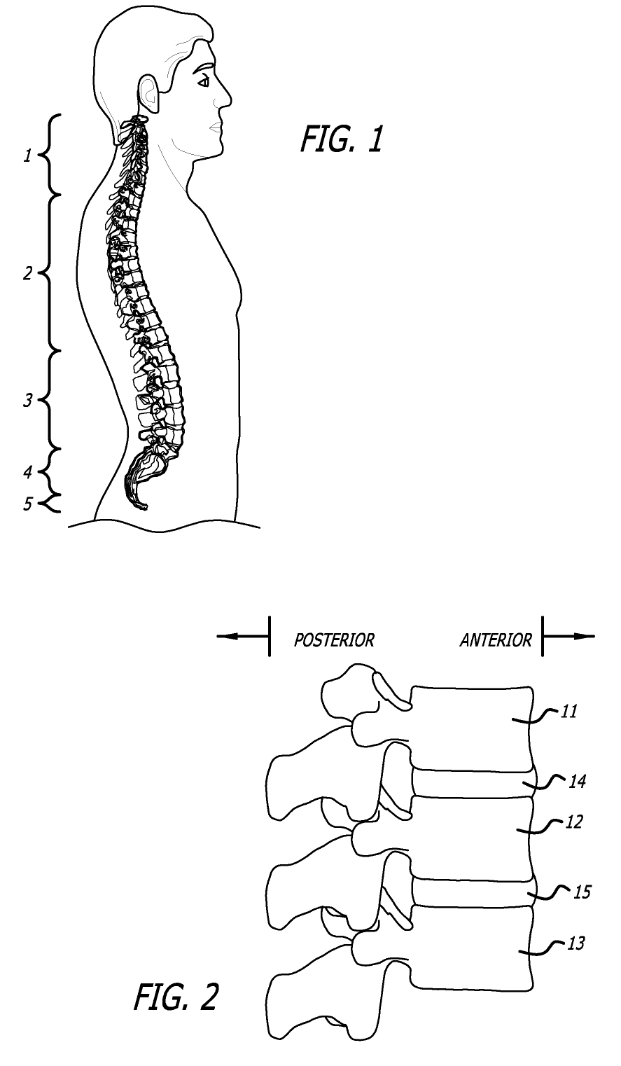

Human spines (FIG. 1) have seven cervical vertebrae 1 in the neck, twelve thoracic vertebrae 2 below the neck, five lumbar vertebrae 3 of the lower back, one sacrum 4 below the lumbar region and one coccyx 5.

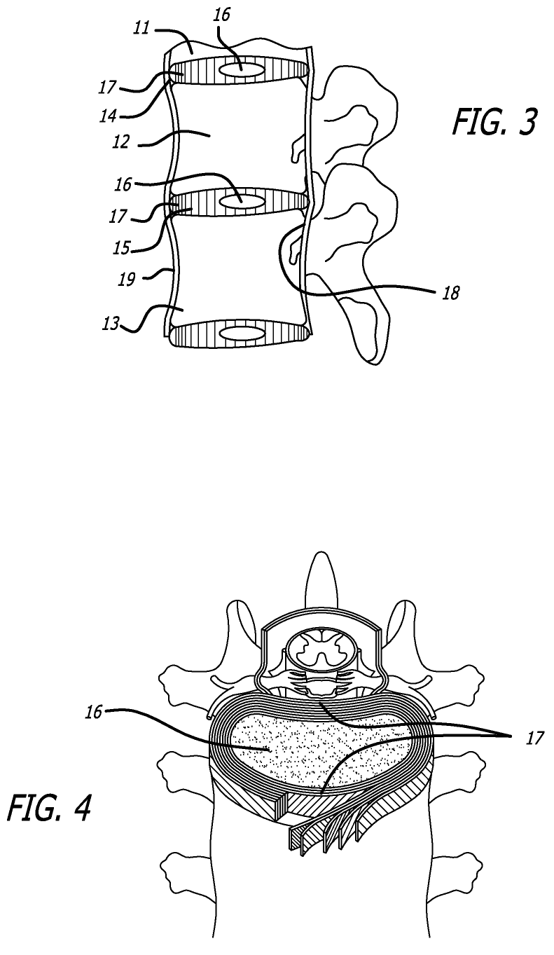

Intravertebral discs 14 and 15 separate adjacent vertebral bodies 11, 12 and 13 (FIGS. 2 and 3). Each disc has a nucleus pulposus 16 surrounded by an annulus fibrosus 17. See also FIG. 4. FIG. 3 also shows the posterior longitudinal ligament 18 and the anterior longitudinal ligament 19, which secure the vertebrae and disc together. Other ligaments also are present but are not discussed.

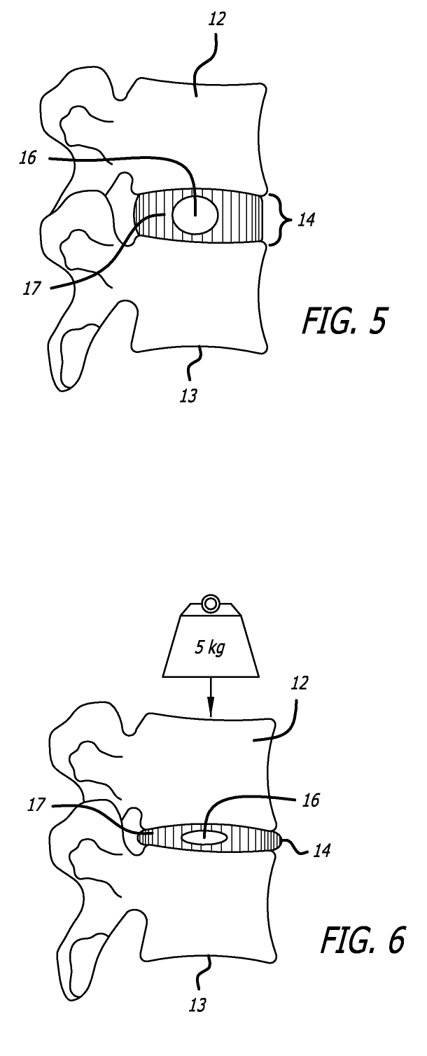

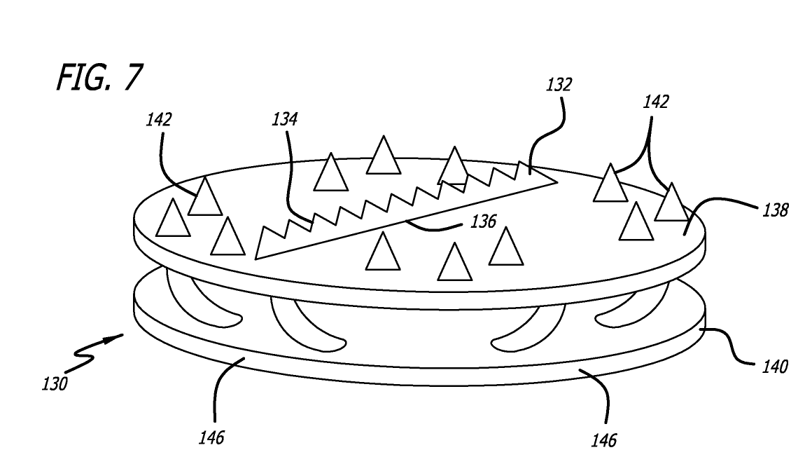

The representation of a disc in FIG. 4 shows the nucleus pulposus 16 surrounded by the outer annulus fibrosus 17. The annulus fibrosus acts as a constraining ring primarily composed of collagen. It allows the intervertebral disc to rotate or bend without significantly affecting the hydrostatic pressure of the nucleus pulposus. The nucleus pulposus consists of proteoglycan, which has an affinity for water molecules. The water hydrates the nucleus pulposus. The hydrated nucleus generates hydraulic effects to act as a shock absorber for the spine. FIGS. 5 and 6 show that effect. Heavy loads applied to adjacent vertebrae 12 and 13 compresses disc 14. See FIG. 6. The nucleus pulposus becomes loaded, but is only slightly compressible. Therefore, the force is transmitted to the annulus fibrosus, which are tensioned. The bands of the annulus fibrosus stretch to absorb the force and then contract to their original length when the load releases.

The FIG. 3 pattern continues to the adjacent vertebrae. Each vertebra is different from its adjacent vertebra, however. For example, lumbar vertebrae are larger than thoracic ones. See FIG. 1. In addition, cervical and lumbar discs are thicker anteriorly, which contributes to lumbar lordosis, or spinal curvature. Thoracic vertebrae are more uniform.

Natural intervertebral discs can be replaced with disc prostheses. Applicant replaces natural intevertabral discs with a system that uses one or more springs to restore more natural anatomical spinal disc support function. The spring material preferably is titanium or cobalt-chromium-molybdenum alloy. The latter, called “cobalt-chrome” (CoCr) often contains other elements in smaller quantities. Titanium and CoCr are the most common metals used inside the body because of their strength and resistance to wear, corrosion and biological activity. Other materials such as plastics and ceramics likely lack the proper mechanical and chemical stability for the environment. Insofar as the application refers to bio-compatible material, it refers to CoCr, titanium or other sufficiently strong and rugged material.

Conventional coil springs are formed of spring wire wound into a coil. The spring wire typically is circular with a constant diameter along the length of the spring coil. This application describes the use of springs that are formed differently. Nevertheless, this application refers to the component that forms the spring as spring wire.

The spring or springs have several configurations including an hourglass and conical configurations. See the shapes in applicant's Ser. No. 11/027,728, which are incorporated by reference. Those springs may be symmetrical or asymmetrical about the spring's longitudinal axis with constant or varying cross-section areas. Note that those springs have multiple turns. The choice of the number of turns is discussed below. However, optimizing the spring or springs may require testing for different spinal parameters. The spring may have less than one turn, or it may have multiple turns. The exemplary embodiment discussed below has two springs, each about % of a turn and offset from each other.

The prosthesis's spring and any associated elements should fit in the space vacated by the replaced natural disc, which usually varies between 5 and 8 mm. Attempting to alter the space changes all other spinal dimensions and usually should be avoided.

Having adjacent spring or adjacent sections of the same spring contact each other, even slightly, when the spring compresses creates friction and sound that detract from performance. In addition, when springs contact each other, microscopic amounts of metal may break off from the spring. Migrating metal particles might create biological reactions that may weaken adjacent tissue such as the spinal nerves in the vertebrae.

The hour-glass spring design provides greater support stability at the vertebrae bone contact regions compared to the conical design which has less contact footprint at one end. For the hour-glass spring structure which has one cross-over, proper design avoids contact by limiting compression travel. The conical spring design allows adjacent turns to compress without contact but is imbalanced in terms of the end contact footprints. The latter requires other end contact support such as end plate to provide the desired balanced support.

Natural discs undergo various motion involving six degrees of freedom including compression, torsional, flexion and combinations of such movements. However, the disc movement and deflections are quite constrained even with large external static and dynamic loads. Compression loading is one of the most important, and in the lumbar region dynamic loading can exceed 1 to 2 kN/mm of force with normal human discs compressing 0.1 to 0.3 mm. Any spring-based prosthesis should limit its potential compression under full load to that range. Factors including the spring configuration, the diameter, configuration and material of the spring wire, the anticipated load, the available space between the vertebrae, the number of springs used in the prosthesis, and other factors. Applicant believes that a spring disc system that fits within a 5 to 8 mm space and deflects 0.2 and 0.3 mm with such loading probably can have no more than one turn and should be formed of thick spring material with varying cross-sectional area along the spring turn length. To provide balance stability, multiple springs with ends terminating onto end plates are needed. The end plates would have fixation features such as spikes and keels to secure to the spine vertebrae surfaces with the end plates having flat to varying surface contours. However, applicant discovered that turning such springs from CoCr spring wire and repeatedly holding tolerance is very difficult. In addition, the incorporation of end plates with such fixation features to the multiple spring ends requires welding techniques. Welding necessarily creates weld joints, which are potentially susceptible to fatigue crack failure associated with cyclic loading encountered by spinal discs.

Single-piece or “monolithic” prostheses that allow custom parameters for different height and weight persons, for different vertebrae and for different disease and damage are desirable, especially from the surgical implant procedure. With the spring winding difficulties of CoCr for the required parameters, using custom wound CoCr springs to accommodate the anticipated loads for the different prostheses dimensions would be particularly difficult, especially for a reasonably prompt turnaround, Welding of such springs to end-plates, which could be technologically viable, still poses manufacturing control problem where a microscopic defect could lead to fatigue failure.

Computer-controlled, robotic micro-machining (computer numerical control or “CNC”) using routing, milling, grinding or other metal removing tools is applicant's principal way to form most or all of the prosthesis including the spring or springs. Such a process allows for an idealized customized prosthesis that can vary for individual patients (e.g., a 1.9 m male or a 1.6 m female) and the different locations on the spine (e.g., between lumbar and thoracic vertebrae or between L4-5 and L2-3) taking into account the patient's weight and loading support requirement. Thus, before the surgical team begins the fabrication process, the patient must be measured to analyze his or her existing vertebra and discs that include size, shape and loading (combined degrees of freedom) requirements. Computer-aided design, modeling and simulation can define a custom monolithic spring disc system that satisfies the patient “form, fit, function” requirements. The measured and modeled data generated for the patient's disc replacement requirement is converted into computer program code for the computer control for the robotic CNC micro-machining of the prosthesis. This results in a patient-customized monolithic prosthesis based on the parameters obtained during the earlier measurement.

The surgeon, his or her staff or colleagues begins the process by imaging (X-ray, MRI, CAT scan, etc.) a patient's diseased or damaged disc and the surrounding region. The more sophisticated imaging tools provide more accurate information about the surrounding vertebra intervertebral disc space. The imaging data combines with personal information such as gender, age, weight, fitness, other disease and earlier spinal surgery to generate a size, and shape (end-plate shape, surface contour, disc thickness and other parameters) and load requirements with associated degrees of freedom for a prosthetic spring disc system. When considering prosthesis requirements, the surgeon also considers the location where the prosthesis will be inserted. Cervical, thoracic or lumbar prostheses likely have different forms, fit differently and perform their function differently.

The analysis also requires surgeons to consider the size and shape of the adjacent vertebrae following removal of the diseased disc. They consider the spacing and the shape of the facing vertebral surfaces and any damaged bone requiring removal. Because of the available shapes and dimensions of applicant's prosthesis, the surgeon may choose particular shapes for the vertebral surfaces and any damaged bone requiring removal. Those surfaces may be flat, but the surgeon could make them concave or some other shape or contour. The disease or other conditions of the vertebra and the patient's overall health may affect the surgeon's decisions. Experiment and computer-aided design and modeling are used to specify the parameters.

The patient analysis requires loading analysis including weight and anticipated loading in various degrees of freedom. The prosthetic spring disc system's effective “spring constant” must be considered with custom definitions. The spring constant may need to be experimentally derived or modeled (numerically simulated with finite element analysis techniques) for varying turn diameters or varying spring cross-sectional area spring structures.

A surgeon also may use the same patient imaged intervertebral profile to perform robotic disc space preparation to match the prosthetic disc. Even if the disc space is not prepared robotically, the surgeon still can use the data for conventional surgery.

Applicant appreciates that with imaging and modeling techniques, custom prostheses can be created. Nevertheless, some patients may be “standard” such that the surgeon may use one of several standard prostheses from an inventory of this application's prostheses.

Once the surgeon and staff complete the analysis, the analyzed data is used to design the prosthesis. The design accounts for thickness, overall shape and size, spring configuration, the effective spring constant and the end-plate surfaces that engage vertebrae. The design also may vary how the prosthesis attaches to the vertebrae via fixation and stabilizing structures including spikes, keels and porous bone in-growth approaches. If the design allows for different materials (titanium, CoCr or another material), the design also must account for the material.

Each parameter affects the design. For example, assume that a patient needs a prosthesis of a certain height and spring constant. The design must account for a spring design that provides the spring constant fitting within the height. That design must account for the spring wire thickness, coil diameter and number of coils, keeping the following equation for determining the stiffness, k, of a spring:

$$k = \frac{{d}^{4} G}{8 {D}^{3} N} \qquad ( 1 )$$

where d is the wire diameter;
G is the shear modulus of the material;
D is the spring diameter; and
N is the number of coils.

The equation assumes the spring wire is circular and has a constant diameter, and the coil(s) is a circular helix. The spring constant must account for elliptical, polygonal or other shaped spring wire or wire that varies over its length if the prosthesis uses these variations. Likewise, the constant must account for different shaped coils. Computer-aided design modeling and simulation is used to estimate the effective spring constant of the patient custom spring disc system that implements the varying dimensional and shape structure from the ideal spring.

In addition, the best arrangement may use two or more disc prostheses spaced about adjacent vertebrae's surfaces. For example, having two separate, laterally spaced monolithic prostheses would spread the spring force to the sides of the vertebrae. Depending on the patient's other health problems, having one prosthesis with different parameters than the other prosthesis may help treat the other problems. Using two smaller prostheses instead of using one larger prosthesis may make surgical insertion easier, such as inserting posteriorly rather than laterally respect to the spinal column.

The exemplary embodiments do not use conventionally wound spring wires. Instead, the prosthesis including the spring or springs with monolithically-integrated end plates with fixation features such as spikes and keels is micro-machined from a block of CoCr, titanium or other biocompatible material. The data obtained from analysis generates instructions for the computer-controlled micro-machining of the block. The data is used to determine the shape, size and loading characteristics of the prosthesis.

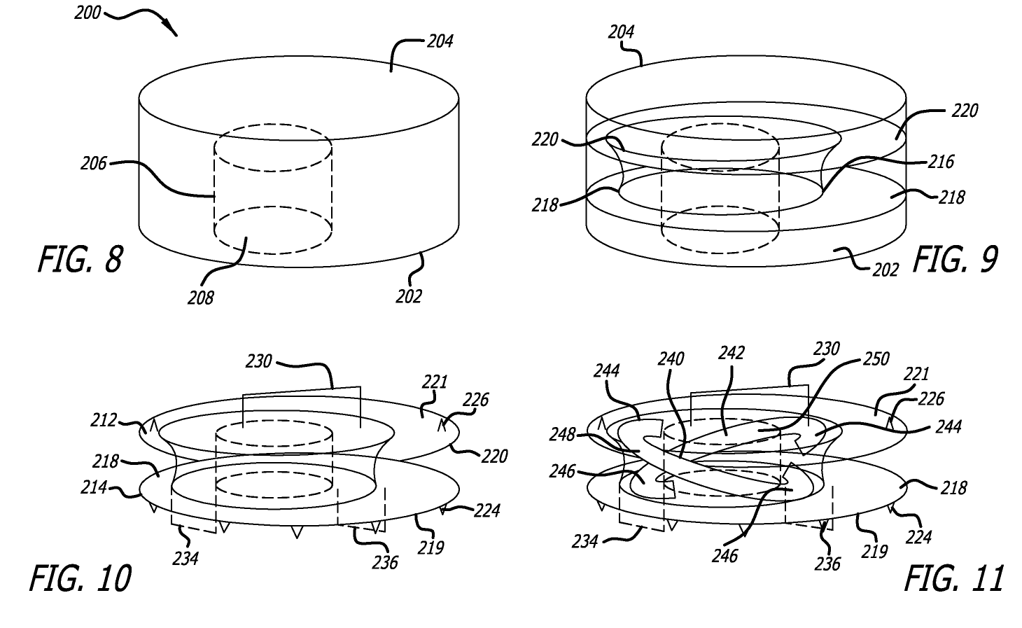

Block 200 in the exemplary embodiment (FIG. 8) may be cut from a larger rod, or the fabricator may receive it as a block. The block may be elliptical, circular or another shape. Though one could use rectangular or polygonal shapes, rounded ones are preferred. The top 204 and bottom 202 are initially parallel in the exemplary embodiment, but the block could start with angled faces or be machined to change the angle of the faces.

The fabricator machines a hollowed-out cavity 206 (FIG. 11). The cavity is open at the bottom 202 of the block to form an opening 208. The cavity does not extend through the top 204, although some end-plate designs could permit the cavity to penetrate the top. Although the cavity is said to be in the bottom of block, it could be cut in the top instead. Conventional machining techniques with a mill or other tool is the preferred way to form cavity 206, but one can form the cavity by micro-machining. In addition, the prosthesis fabricator could obtain blocks with an existing cavity. In the exemplary embodiment, the cavity is off center and circular, but it may be elliptical or have another shape.

Most of the remainder of the fabrication process uses computer-controlled, micro-machining techniques based on instructions generated during measurements of and other information about the patient. The exemplary embodiment uses robotic micromills, routers, grinders and other micro metal removing tools. Typically, they are similar to dental tools and will use diamond-tipped or tungsten carbide heads and remove small, controlled amounts of material. Applicant contemplates using electro-etching, lasers and other techniques but believes that for the fabrication of the detailed structure of applicant's prosthesis, micro-milling, routing and grinding under robotic control is preferred. Nevertheless, for some parts of the structure, the process may combine micro-machining with other metal-removing processes.

Using micro-machining, the tool(s) cuts from the outside of the block and forms sidewall 216 extending between the lower and upper planes 218 and 220 respectively. Those planes will become surfaces that contact the adjacent vertebrae. The taper of sidewall 216 is asymmetric in the exemplary embodiment in that the diameter at the upper end 220 is greater than the diameter at the lower end 218.

The process machines away part of the bottom and top 202 and 204 (FIG. 9), but the machining leaves fixation structures in the bottom and top of the block including spikes 224 and 226 in the top and bottom outer surfaces 221 and 219. FIGS. 10 and 11. Accordingly plate 212 forms between surfaces 220 and 221, and plate 214 forms between surfaces 218 and 219. To form the spikes, the mill or other tool (such as electro-etching) removes the material around each spike, which allows the spike to protrude above the top or bottom surface. Alternatively, the outer surfaces may be milled flat and the spikes could be deposited on the surface.

The upper and lower surfaces 220 and 218 are flat and parallel in the FIGS. 10 and 11 exemplary embodiments. However, insofar as the surgeon chooses to have angled surfaces or concave, convex or different contoured surfaces, the micro-machining process can generate those surfaces.

Similarly, the process also forms a keel 230 extending upward from top 220. The keel may have serrations or other features (not shown) for engaging bone on the vertebra above the prosthesis. Two spaced-apart bottom keels 234 and 236 extend down from bottom 218. In FIG. 11, the bottom keels are near the outside of the bottom plate 218, but they can be in different positions. Moreover, though the exemplary embodiment has two such keels, the prosthesis could have one, three or more. The exemplary embodiment uses multiple, spaced-apart bottom keels because opening 208 of cavity 206 likely prevents the forming of a more centered keel such as keel 230 using the fabrication process being discussed.

The keels cooperate with the spikes and any other fixation structures to secure and stabilize the prosthesis to adjacent vertebrae. Keels may be particularly important when a patient bends over. Bending causes the anterior portions of adjacent vertebrae to move toward each other while the posterior portions move apart. Forces on the prosthesis tend to push the prosthesis in the posterior direction. One or more properly sized and placed keels resist those forces.

The upper keel 230 may be aligned or angled to lower keels 234 and 236. Ideally, the keels are positioned so to resist particular forces between the vertebrae and the prosthesis. In the exemplary embodiment, keel 230 mounts at a 45° angle.

The surgeon also can use adhesive or a porous ingrowth surface coating on the plates and the spikes to secure the section to the vertebral bodies. Doi, U.S. Pat. No. 5,541,184 (1996) is one of many patents showing the use of porous ingrowth the coating for prostheses (a hip prosthesis).

The next step is micro-machining to form the springs. The loading charactistics of the springs is determined from the data obtained from the patient profile and imaging of the damaged or diseased disc and surrounding vertebrae. The FIG. 11 embodiment has two springs, 240 and 242. The micro-machining removes material around the eventual springs 240 and 242 and leaves material to act as springs. Although this description forms the springs last, the order of machining various structures may be varied where appropriate. Micro-machining also can remove material to form two or more features simultaneously.

Spring 240 has an upper end 244 and a lower end 246 at the end of spring body 248. Likewise, spring 242 has an upper end 244 and a lower end 246 at the end of spring body 248. By micro-machining through the bottom 208 of cavity 206, the machining forms the springs in their desired shape to provide the form and function for the particular prosthesis based upon chosen parameters.

The two springs 240 and 242, which each extend between about ¾ and ½ turn in the exemplary embodiment (FIG. 11), are examples only. Each spring has the shape of a coarse spiral. Applicant believes that the diameter of each spring is springs 240 and 242 is greater near the top and bottom and smaller near the center (quasi-conical). The smaller diameter is still relatively large to provide the necessary spring constant for this environment. Using conventional spring winding techniques with CoCr or titanium wire of these short lengths, large wire diameter and tight winding is very difficult if not impossible using current technology.

Other configurations are possible. In FIG. 11, one spring extends from about 45° from the anterior of the prosthesis to about 45° from the anterior. The other spring is about 90° from the other spring. This arrangement spreads the compressive force more evenly to the upper and lower surfaces 220 and 218. Intersecting the springs near the periphery of upper and lower surfaces probably yields more stable fixation to the adjacent vertebral surfaces because the springs' starting and end points are further apart.

The micro-machining that forms the springs 240 and 242 leaves material near the intersection of the top and bottom plates 212 and 214 with the respective ends 244 and 246 of spring 240 and respective ends 252 and 254 of spring 242. Thus, the springs remain attached to the top and bottom plates. An abrupt transition between the springs and the plates could become an area of potential weakness, but the micro-machining process can machine a smooth transition.

The order that computer control machines parts of the monolithic prosthesis may vary considerably. It may allow machining of two or more parts of the prosthesis simultaneously. That is, cutting from the inside of cavity 206 can occur while one or more cutting tool cuts into the outside of the block,

Applicant contemplates that a single spring in an hourglass or conical configuration could be used if the parameters call for such a spring. These configurations probably would have fewer turns than the springs in applicant's earlier application. Instead of having 2½ or more turns, the embodiments would have two or fewer turns. With such few turns, the differences between hourglass and conical configurations may become unimportant. Micro-machining allows the springs to have any useful shapes as long as the springs have the proper spring constant and perform their proper functions.

Springs wire that forms conventional springs generally has a constant diameter, but micro-machining allows the spring wire to have a varying shape and diameter. Equation (1) shows that spring stiffness is directly related to wire diameter. Thus, one could have non-linear response from the spring by having a non-constant wire diameter For example, the spring could be more compressive under moderately elevated loads but resist compression more when greater loads are applied. Thus, the design can facilitate softer compressive mode in the unloaded equilibrium state.

In addition, using the shapes of springs in FIG. 11, fitting three or more such springs between plates 212 and 214 may be possible as long as they do not contact each other under load. Similarly, even shorter springs could be spaced along the periphery of the plates. Applicant's previous application has embodiments using six such springs.

After the process is completed, the finished prosthesis is heat treated to anneal stress defects. The prosthesis also is electro-polished. Inspection including load testing and visual and X-ray inspections are performed to check performance and quality control.

Whether plates 212 and 214 are flat, curved or have another contour, the surfaces can deviate from being parallel with angles between about 6° and 12° to accommodate natural disc spacing shapes, especially in the lumbar regions. Spring coils of different lateral thicknesses with appropriate spring constants would facilitate the non-parallel plate requirements.

The surgical procedure discussed below briefly tracks the general steps, but this brief explanation of the surgery does not discuss all the detail of a complex procedure. Skilled surgeons understand the steps, however. Applicant contemplates that the surgeon will use manual techniques for most or all the surgery. However, parts of the surgery may be under computer control based on instructions generated from the initial patient data and from the computer controlled micro-machining process.

The surgery for conventional prostheses usually begins with cutting the vertebral surfaces to conform to the prosthesis that the surgeon inserts. Minimizing the amount of bone removed is advantageous. In addition, the surgeon usually accesses the space between adjacent vertebrae from the side. However, a side or lateral access requires going through substantial amounts of tissue. Applicant's prosthesis can be formed to conform to the vertebral surfaces that remain after removing the patient's natural disc and cleaning the vertebral surfaces. Therefore, applicant's prosthesis may allow for less invasive surgery on the remaining vertebral bone.

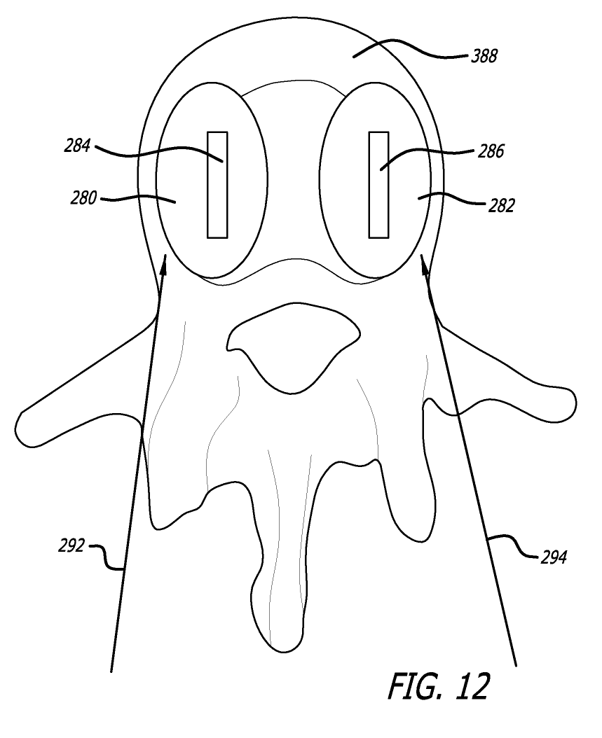

After exposing the intervertebral region, the surgeon secures the vertebrae apart and removes the existing disc. In the lumbar region, small spaces to the sides of the transverse processes allow a more posterior access to the disc and intervertebral space. See FIG. 12. The surgeon can remove the disc though the spaces and can cut, grind or otherwise prepare the bone of the vertebrae to yield a clear prosthesis receiving opening for the prosthesis. Some of the surgical steps may use robotic tools for more precise cutting and disc placement.

Tools spread the vertebrae far enough apart for inserting the disc prosthesis. Because of the additional height that that the fixation keels and spikes provide, the vertebrae must be pushed far enough apart to accommodate the fixation structure. The surgeon inserts the disc or discs manually or robotically. The patient's previously imaged intervertebral profile can be used to perform robotic disc space preparation to match the prosthetic disc.

Alignment and placement are checked visually and by X-ray, MRI or CAT scan.

Having two or more laterally spaced discs may offer advantages. FIG. 12 shows two such discs 280 and 282. Each has a keel 284, 286 extending upward from a vertebra's top surface 288. Positions of the discs may vary. The discs may be small enough to allow them to be inserted posterior between small spaces on the sides of the trans-verse processes in the direction of arrows 292 and 294. Posterior insertion can minimize damage to tissue surrounding the vertebrae that lateral entry may cause.

The foregoing is merely illustrative and not limiting, having been presented by way of example only. Although examples have been shown and described, it will be apparent to those having ordinary skill in the art that changes, modifications or alterations may be made.

Although many of the examples presented involve specific combinations of method acts or system elements, those acts and elements may be combined in other ways to accomplish the same objectives. Acts, elements and features discussed only in connection with one embodiment are not intended to be excluded from a similar role in other embodiments.

“Plurality” means two or more. A “set” of items may include one or more of such items. As used in this application, whether in the written description or the claims, the terms “comprising,” “comprised of,” “including,” “carrying,” “having,” “containing,” “involving,” and the like are to be understood to be open-ended, i.e., to mean “including but not limited to.” Only the transitional phrases “consisting of” and “consisting essentially of,” respectively, are closed or semi-closed transitional phrases with respect to claims.

Use of ordinal terms such as “first,” “second,” “third,” etc., in the claims to modify a claim element does not by itself connote any priority, precedence or order of one claim element over another or the temporal order in which acts of a method are performed. The terms are merely labels to distinguish one claim element having a certain name from another element having a same name (but for use of the ordinal term) to distinguish the claim elements.

I claim:

1. A prosthetic single-piece or monolithic spinal disc for replacing a damaged or diseased natural disc within a prosthesis receiving opening between opposed surfaces of two adjacent vertebrae, the vertebrae having a common longitudinal axis, wherein the prosthetic spinal disc comprises:
a) a fitting of a chosen material sized to be received within the prosthesis receiving opening, the fitting having an upper plate and a lower plate movable relative to each other,
b) each upper and lower plate having an outer surface, at least part of the outer surface of the upper and lower plates of the fitting conforming to the opposed surfaces of the prosthesis receiving opening;
c) at least one spring received within a space between the upper and lower plates, the spring biasing the upper and lower plates apart,
d) wherein the fitting is formed by the following process:
i) providing a block of the chosen material; and
ii) using at least one micro-machining tool, cutting into the block to form the upper and lower plates and the at least one spring and discarding any unused material from the block.

2. The prosthetic spinal disc of claim 1 wherein the process further comprises forming the fitting's dimensions and the at least one spring based upon measurement of at least the two adjacent vertebrae and the damaged or diseased disc between the two adjacent vertebrae.

3. The prosthetic spinal disc of claim 1 wherein the process further comprises forming the fitting's dimensions and choosing the spring constant of the at least one spring based upon load measurement or modeled information of at least the two adjacent vertebrae and the damaged or diseased disc between the two adjacent vertebrae.

4. The prosthetic spinal disc of claim 2 further comprising forming fixation structure in at least one of the outer surfaces, the fixation structure engaging the opposed surface of the prosthesis receiving opening.

5. The prosthetic spinal disc of claim 1, wherein the block has a top, bottom and side wall, the process for forming the fitting further comprises forming an cavity in the bottom of the block, removing material from the top and bottom of the block to form top and bottom plates, cutting from the cavity to remove material to form the at least one spring.

6. The prosthetic spinal disc of claim 5 wherein the at least one spring has a varying dimension.

7. A process for forming a prosthetic spinal disc adapted to fit between opposed surfaces of two adjacent vertebrae, comprising:
a) providing a block of the chosen material; and
b) using a micro-machining tool, cutting into the block to form an upper plate and a lower plate, the upper plate having an outer and inner surface and the lower plate having an outer and inner surface, the micro-machining tool forming at least one spring between the upper and lower plates to bias the upper and lower plates apart.

8. The process of claim 7 wherein the block has a top and bottom, the cutting step further comprises forming a cavity in the bottom of the block, machining upper and lower surfaces and forming a sidewall between the upper and lower surfaces, forming the at least one spring within the sidewall.

9. A process for creating a prosthesis of a particular size, shape and loading for replacing a damaged or diseased intervertebral disc comprising:
a) generating a patient profile from information about the patient and the disease or damaged disc and adjacent vertebrae and generating a measured profile based on imaging the damaged or diseased disc and adjacent vertebrae;
b) using a computer-based model for defining the prosthesis single and multiple spring structures (spring size, shape, turns and other factors) to provide the necessary dynamic load support (in various degrees of freedom) as required anatomically (e.g. 1-2 kN/mm with 0.2-0.3 mm deflection for lower lumbar).
c) generating instruction for computer controlled machining of a block of biocompatible material based on the patient's measured and modeled prosthesis information; and
d) removing material from the block using micro-machining under computer control from the instructions to form the prosthesis from the block including forming a top and bottom plate and at least one spring between the top and bottom plates.

10. The process of claim 9 wherein the step of generating a patient profile comprises obtaining information about the patient's sex, height and weight and the location of the damaged or diseased disc.

11. The process of claim 9 wherein the step of generating a measurement profile comprises imaging the damaged or diseased disc and the vertebrae adjacent the damaged or diseased disc.

12. The process of claim 9 wherein the step of generating spring design information comprises computer-aided design based on patient load support profile and numerical modeling/simulation of the spring constant to achieve the necessary anatomical performance (disc load/deflection).
